(19) United States
(12) Patent Application Publication
(10) Pub. No.: US 2007/0225799 A1
(43) Pub. Date: Sep. 27, 2007

(54) STENT, INTRALUMINAL STENT DELIVERY SYSTEM, AND METHOD OF TREATING A VASCULAR CONDITION

(75) Inventor: David Doty, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., SANTA ROSA, CA (US)

(21) Appl. No.: 11/277,419

(22) Filed: Mar. 24, 2006

(51) Int. Cl. A61F 2/06

(52) U.S. Cl. 623/1.38

(57) ABSTRACT

A stent, a stent delivery system, and a method of treating a vascular condition. The system includes a catheter, an inflatable member operably attached to the catheter, and a biodegradable stent disposed on the inflatable member. The stent includes a biodegradable flexible elongate member including an elongate member wall surrounding a cavity. A biodegradable reinforcing member is positioned within or adjacent the elongate member wall to support the biodegradable sleeve. A biodegradable photo-curable polymer positioned within the cavity. The method includes delivering a biodegradable stent having a cavity filled with a pre-polymer to a treatment site. A balloon is expanded to position the stent at the treatment site. The pre-polymer positioned within the stent cavity is photopolymerized. The deployed stent is supported in a radial direction with a biodegradable reinforcing member.

### TECHNICAL FIELD OF THE INVENTION

The present invention relates generally to stents. More particularly, the invention relates to a stent, an intraluminal stent delivery system, and a method of treating a vascular condition.

### BACKGROUND OF THE INVENTION

Balloon angioplasty has been used for the treatment of narrowed and occluded blood vessels. A frequent complication associated with the procedure is restenosis, or vessel re-narrowing. Within 3-6 months of simple angioplasty, restenosis can occur in about half of patients. To reduce the incidence of re-narrowing, several strategies have been developed. Implantable prosthetic devices, such as stents, have been used to reduce the rate of angioplasty related restenosis by about half. The use of such prosthetic devices has greatly improved the prognosis of these patients.

The objective in angioplasty is to enlarge the lumen of the affected coronary artery by radial hydraulic expansion. This is generally accomplished by inflating a balloon within the narrowed lumen of the affected artery. Radial expansion of the coronary artery may occur in several different dimensions, and is related to the nature of the plaque. Soft, fatty plaque deposits are flattened by the balloon, while hardened deposits are cracked and split to enlarge the lumen. The wall of the artery itself may also be stretched as the balloon is inflated. With simple angioplasty, the balloon may be threaded through the artery with a catheter and inflated at the place where the blood vessel is blocked. After the procedure, the balloon is then removed. The stent may then be used to support open the artery. The stent may be deployed along with the balloon or after the balloon is removed.

The stent may be formed from a generally tubular body that can be expanded from a collapsed state into a deployed state. The stent body may include a plurality of elongated element lengths (e.g., wire lengths, or the like) that are connected together to permit the stent body to be expanded. The stent may be coupled to a deployment system (e.g., a catheter) in a collapsed state. For example, the stent may be compressed within a lumen formed within a catheter or onto a catheter balloon. The catheter including the stent may be then advanced endovascularly (or within another vessel type) to the afflicted region of the body passage. While fed through the vessel, the stent remains in the collapsed state.

Once the stent has reached the afflicted region in the body passage, it may be expanded radially outward into the deployed state. The stent may be expanded into its deployed state by inflating the catheter balloon so that expansion of the stent is achieved simultaneously with the inflation of the balloon. Alternatively, the stent may be manufactured from a resilient material such that when it is collapsed, the stent may naturally expand from a “tense” collapsed state into a “relaxed” deployed state. In such a case, the stent self-expands as it is removed from the catheter lumen. Regardless of the type of stent, the radial strength of the stent should be sufficient to withstand restenosis in order to maintain vascular patency. Certain stents (e.g., non-metallic, bioabsorbable types) lack sufficient radial strength under stressful conditions (e.g., high blood pressure, bodily movements, etc.). As such, it would be desirable to provide a bioabsorbable stent with an improved radial strength.

Given that the stent deployment system typically includes a number of parts, a reduced collapsed stent profile size contributes to a reduced size in the deployment system. As such, numerous benefits may be provided by a reduction in stent and (potentially) deployment system size. For example, as the stent is advanced to the site of deployment, it may encounter a sometimes tortuous and narrow network of vessels. Smaller sized stents and deployment systems may facilitate easier negotiation of such vessel networks. Other benefits of reducing the size of the deployment system may include less disruption of an atheroma and plaque that could lead to emboli, less disruption of blood flow, less likelihood of vessel wall damage, and reduced vessel puncture size for intraluminal access. Accordingly, it would be desirable to minimize the stent collapsed profile size.

Accordingly, it would be desirable to provide a stent, an intraluminal stent delivery system, and method of treating a vascular condition that would overcome the aforementioned and other disadvantages.

### SUMMARY OF THE INVENTION

A first aspect according to the invention provides a stent. The stent includes a biodegradable flexible elongate member including an elongate member wall surrounding a cavity. A biodegradable reinforcing member is positioned within or adjacent the elongate member wall to support the biodegradable elongate member. A biodegradable photo-curable polymer positioned within the cavity.

A second aspect according to the invention provides an intraluminal stent delivery system. The system includes a catheter, an inflatable member operably attached to the catheter, and a biodegradable stent disposed on the inflatable member. The stent includes a biodegradable flexible elongate member including an elongate member wall surrounding a cavity. A biodegradable reinforcing member is positioned within or adjacent the elongate member wall to support the biodegradable elongate member. A biodegradable photo-curable polymer positioned within the cavity.

A third aspect according to the invention provides a method of treating a vascular condition. The method includes delivering a biodegradable stent having a cavity filled with a pre-polymer to a treatment site. A balloon is expanded to position the stent at the treatment site. The pre-polymer positioned within the stent cavity is photopolymerized. The deployed stent is supported in a radial direction with a biodegradable reinforcing member.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 illustrates an intraluminal stent delivery system in accordance with the present invention;

FIG. 2 illustrates a stent in accordance with the present invention;

FIG. 2A illustrates a cross section of a portion of the stent illustrated in FIG. 2;

FIG. 3 illustrates a cross-section of the stent of FIG. 2 shown deployed in a vessel, in accordance with the present invention; and

FIG. 4 illustrates a flowchart of a method of treating a vascular condition, in accordance with one embodiment of the present invention.

### DESCRIPTION OF THE PRESENTLY PREFERRED EMBODIMENTS

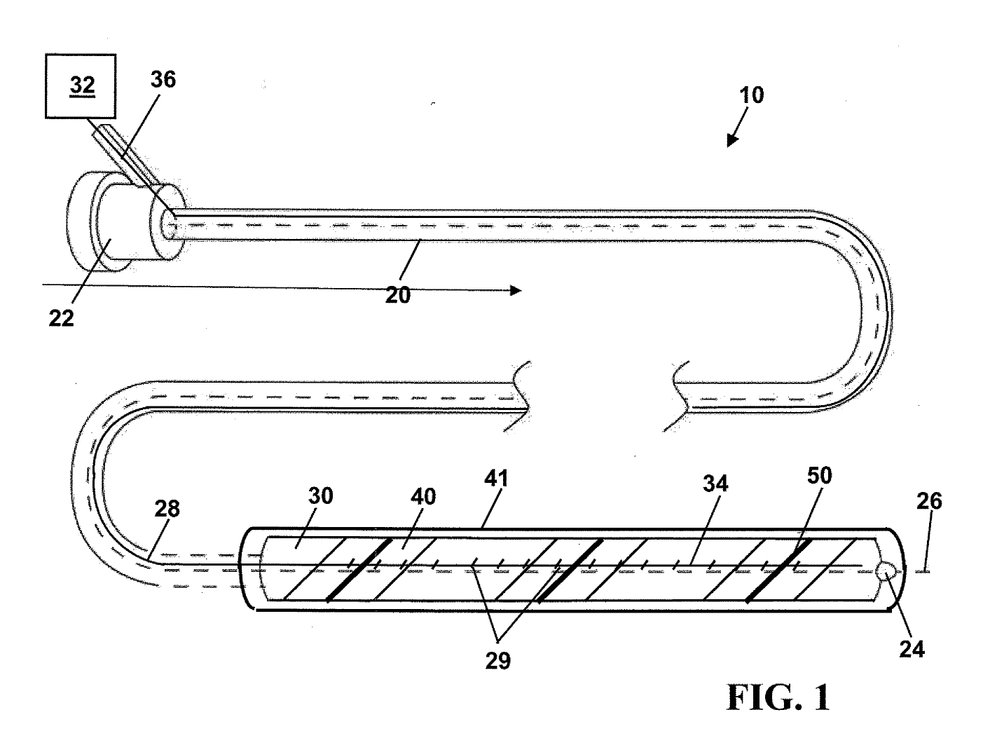

Referring to the drawings, which are not necessarily drawn to scale and wherein like reference numerals refer to like elements, FIG. 1 is a perspective view of an intraluminal stent delivery system in accordance with one embodiment of the present invention and shown generally by numeral 10. System 10 includes a catheter 20, a balloon 30 operably attached to the catheter 20, and a stent 40 disposed on the balloon 30. Stent 40 (shown in a compressed configuration) remains compressed on the balloon 30 during advancement through the vasculature. The compressed stent 40 includes a small profile (i.e., cross-sectional size). In one embodiment, a sheath 41 may be disposed on the stent 40 to protect the stent 40 as well as the vessel walls during advancement.

Although the devices described herein are primarily done so in the context of deployment within a blood vessel, it should be appreciated that intravascular and/or implantable prosthetic devices in accordance with the present invention may be deployed in other vessels, such as a bile duct, intestinal tract, esophagus, and airway.

The term “biodegradable” refers to substances that degrade (e.g., via hydrolysis) to at least a certain extent within the body. Biodegradable substances are biocompatible and preferably incur a reduced inflammatory response. A “radial” direction is one that is perpendicular to the axis of a vessel.

In one embodiment, catheter 20 includes an elongated tubular member manufactured from one or more polymeric materials. In another embodiment, catheter 20 includes a metallic reinforcement element. In some applications (such as smaller, more tortuous vessels), the catheter is constructed from very flexible materials to facilitate advancement into intricate access locations. Numerous over-the-wire, rapid-exchange, and other catheter designs are known and may be adapted for use with the present invention. Catheter 20 can be secured at its proximal end to a suitable Luer fitting 22, and may include a distal rounded end 24 to reduce harmful contact with a vessel. Catheter 20 can be manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® resin, Vestamid® nylon, Tecoflex® resin, Halar® resin, Hyflon® resin, Pellathane® resin, combinations thereof, and the like. Catheter 20 includes an aperture formed at the distal rounded end 24 allowing advancement over a guidewire 26.

Balloon 30 may be any variety of balloons or other devices capable of expanding the stent 40 (e.g., by providing outward radial forces). Balloon 30 may be manufactured from any sufficiently elastic material such as polyethylene, polyethylene terephthalate (PET), nylon, or the like. Those skilled in the art will recognize that the stent 40 may be expanded using a variety of means and that the present invention is not limited to balloon expansion.

In one embodiment, an optical fiber 28 is positioned adjacent the balloon 30 and the distal rounded end 24. The main body of the optical fiber 28 is substantially straight and extends through an inflation lumen of the balloon 30 and along the length of catheter 20, terminating proximally at a connecter arm at the Luer fitting 22. Optical fiber 28 is operably connected to a light source 32 via its proximal portion 36. In one embodiment, the distal portion 34 of the optical fiber 28 adjacent the stent 40 is abraded, as shown by hash marks 29, to diffuse light from the optical fiber 28 in multiple directions.

In another embodiment, the optical fiber 28 is attached outside the catheter or, alternatively, freestanding or as part of another medical device. In another embodiment, the optical fiber 28 may be any material or apparatus capable of emitting light and may be connected to a light source by any means of conveying light to the stent. For example, a light source running under its own power source (e.g., a battery) can be positioned adjacent the balloon 30 and distal rounded end 24.

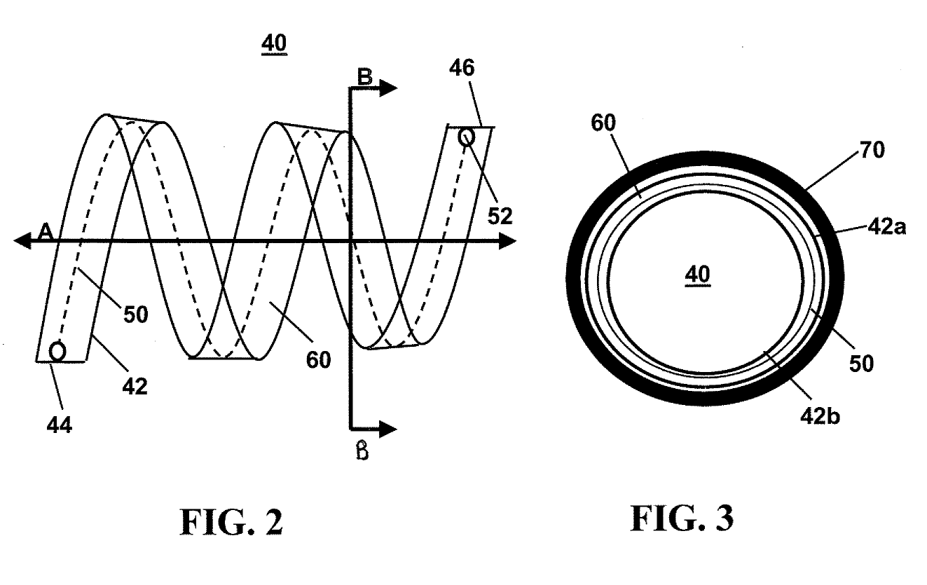

Referring to FIG. 2, an assembled stent 40 in a deployed configuration is shown. In one embodiment, the stent 40 is a generally tubular structure including a passageway that extends along a longitudinal axis A. Stent 40 can be configured for various lengths when in the deployed configuration. In one embodiment, the length of stent 40 is predetermined based on the dimensions of the treatment site.

Stent 40 includes a flexible biodegradable elongate member 42, which is shaped in a coiled configuration. Referring to FIG. 2A, elongate member 42 comprises an elongate member wall 48 forming a cavity 54. In one embodiment, elongate member 42 has a generally oval cross section. In another embodiment, elongate member 42 may have a generally circular cross section. Elongate member 42 can be manufactured from numerous biodegradable materials such as a thermoplastic material of poly-lactic acid (PLA), polyglycolyic acid (PGA), and/or collagen, which demonstrate high biocompatibility with reduced inflammatory response. Those skilled in the art will recognize that the size, geometry, and constituent material of the elongate member 42 may vary from the description and illustrations provided herein.

In one embodiment, a biodegradable reinforcing member 50 is positioned adjacent the elongate member 42. Reinforcing member 50 provides an outward radial force for supporting the stent 40 in the deployed configuration. In one embodiment, reinforcing member 50 is a biodegradable magnesium wire positioned along an inner surface 56 of the elongate member 42. Alternatively, the reinforcing member 50 can be positioned outside or integrated into the elongate member wall 48. In one embodiment, the reinforcing member 50 is manufactured from a resilient material for providing radial force (i.e., to resist restenosis). Reinforcing member 50 is positioned along the length of the elongate member 42 in the coiled configuration. The end portions of the reinforcing member 50 can be shaped to reduce sharp edges. In one embodiment, the end portions form hoops or rings 52. Those skilled in the art will recognize that the size, geometry, number, and constituent material of the reinforcing member 50 may vary from the description and illustrations provided herein. For example, in another embodiment, the reinforcing member may be struts, wires, mesh, and the like, for supporting the stent 40.

In one embodiment, cavity 54 of the elongate member 42 is at least partially filled with a biodegradable, photo-curable polymer 60. The end portions 44, 46 of the elongate member 42 are sealed to retain the photo-curable polymer 60. The end portions 44, 46 may be sealed by any means known in the art such as, for example, thermal sealed, bonded, clipped, and the like, to retain the photo-curable polymer 60. While the stent is in the compressed configuration, mounted on the balloon 30, the polymer 60 is in a fluid pre-polymer form, such as a liquid, gel, and the like. In one embodiment, the pre-polymer comprises a cross-linked hydrophilic polymer, commonly known as hydrogels. In one embodiment, the polymer 60 also includes a photoinitiator, such as eosin Y, which accelerates polymerization of the pre-polymer to the polymer 60. Those skilled in the art will recognize that numerous compounds may be added to the polymer 60 to alter its curative properties.

The pre-polymer liquid contained within cavity 54 is flexible. As such, in one embodiment, mounting of the stent 40 on the balloon 30 is performed with a mechanical wrapping device or other similar device for wrapping the stent in a helical fashion. Mounting the stent 40 tightly around the balloon 30 provides a reduced collapsed profile size.

After the stent 40 is deployed and exposed to light, the liquid pre-polymer polymerizes or “cures” in vivo into the polymer 60. The polymerization of the liquid pre-polymer solidifies the pre-polymer to form the cured polymer 60. The cured polymer 60, along with the reinforcing member 50, supports the elongate member 42 at the treatment site. In one embodiment, the cured polymer 60 and the reinforcing member 50 supports the elongate member 42 in the radial direction to counteract vasoconstriction. The degree of the radial strength and support provided by the cured polymer depends on the nature of the cured polymer 60. For example, an epoxy or acrylic polymeric material may provide greater radial strength than a hydrogel.

In one embodiment, the stent 40 includes at least one therapeutic agent. The therapeutic agent(s) can be applied to one or more portions of the stent 40 including the elongate member 42 and the reinforcing member 50. In other embodiments, the therapeutic agent may be integrated with the polymer 60 thereby allowing elution as the elongate member 42 degrades. It should be noted that any polymer(s) incorporated in a therapeutic agent may or may not be the same as the biodegradable, photo-curable polymer 60.

The therapeutic agent comprises one or more drugs, polymers, a component thereof, a combination thereof, and the like. For example, the therapeutic agent can include a mixture of a drug and a polymer as known in the art. Some exemplary drug classes that may be included are antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like. Specific examples of drugs include abciximab, angiopeptin, colchicine, eptifibatide, heparin, hirudin, lovastatin, methotrexate, rapamycin, streptokinase, taxol, ticlopidine, tissue plasminogen activator, trapidil, urokinase, zotarolimus, and growth factors VEGF, TGF-beta, IGF, PDGF, and FGF.

In one embodiment, the therapeutic agent polymer provides a matrix for incorporating the drug within a coating, or may provide means for slowing the elution of an underlying therapeutic agent when it comprises a cap coat or is incorporated into the photo-curable polymer. It should be noted that the polymer(s) of the therapeutic agent is not necessarily the same compound as the photo-curable polymer 60. Some exemplary biodegradable polymers that may be adapted for use with the present invention include, but are not limited to, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like.

Solvents are used to dissolve the therapeutic agent and polymer to comprise a therapeutic agent coating solution. Some exemplary solvents that may be adapted for use with the present invention include, but are not limited to, acetone, ethyl acetate, tetrahydrofuran (THF), chloroform, N-methylpyrrolidone (NMP), methylene chloride, and the like.

Those skilled in the art will recognize that the nature of the drug and polymer may vary greatly and are typically formulated to achieve a given therapeutic effect, such as limiting restenosis, thrombus formation, hyperplasia, etc. Once formulated, a therapeutic agent solution (mixture) comprising the coating may be applied to the stent 40 by any of numerous strategies known in the art including, but not limited to, spraying, dipping, rolling, nozzle injection, and the like. Numerous strategies of applying the coating in accordance with the present invention are known in the art. In another embodiment, the therapeutic agent may be incorporated within the photo-curable polymer 60.

In one embodiment, two or more therapeutic agents are incorporated into the stent and are released having a multiple elution profile. For example, a first therapeutic agent disposed on the elongate member 42 is released to reduce inflammation. The first agent may be released on a short-term basis to overcome surgical trauma of the treatment. In this embodiment, the second therapeutic agent is disposed in the photo-curable polymer 60 for reducing endovascular restenosis. As the elongate member 42 biodegrades, the second therapeutic agent is released on a longer-term basis.

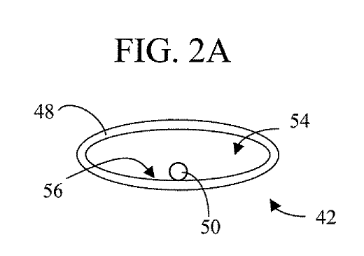

FIG. 3 illustrates the stent 40 in cross-section deployed within a vessel 70, taken along line B-B of FIG. 2. An outer portion of the elongate member 42a contacts an inner wall of the vessel 70. Outer portion 42a of the elongate member 42 is supported by the reinforcing member 50, polymer 60, and inner portion 42b of the elongate member 42, thereby providing axial strength to the stent 40.

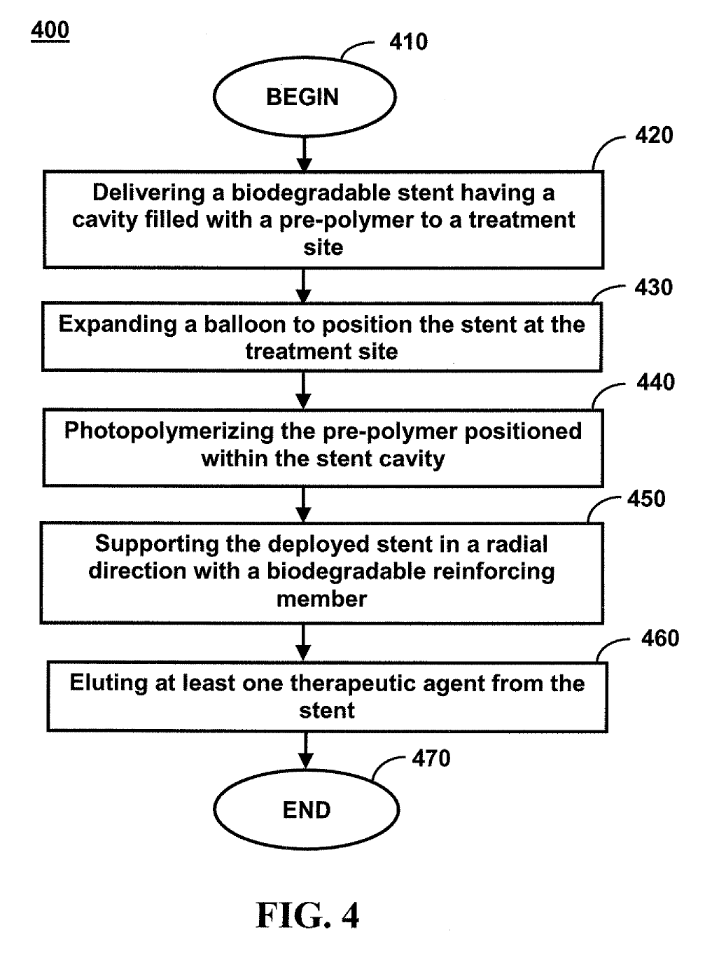

FIG. 4 illustrates a flowchart of a method 400 of treating a vascular condition, in accordance with one embodiment of the present invention. The present description relates to the treatment of a vascular condition, which in this case is an ischemic blood vessel including a vulnerable plaque. The method begins at step 410.

At step 420, the stent 40 is delivered to a treatment site with the catheter 20. In one embodiment, catheter 20 is advanced to treatment site over a pre-positioned guidewire 26. In one embodiment, at least one radiopaque marker may be disposed on the stent 40, catheter 20, and or component thereof to allow in situ visualization and proper advancement, positioning, and deployment of the stent 40. The marker(s) may be manufactured from a number of materials used for visualization in the art including radiopaque materials such as, for example, platinum, gold, tungsten, metal, metal alloy, and the like. Marker(s) may be visualized by fluoroscopy, IVUS, and other methods known in the art. Those skilled in the art will recognize that numerous devices and methodologies may be utilized for positioning an intraluminal stent in accordance with the present invention.

At step 430, once the stent 40 is properly positioned at the treatment site, the balloon 30 and stent 60 are expanded radially into contact with the vessel wall. Balloon 30 is expanded by addition of fluid within its lumen. In one embodiment, a sheath 41, covering stent 40 during delivery to the treatment site, is retracted prior to expanding balloon 30. At step 440, the pre-polymer positioned within the stent is polymerized. In one embodiment, light is passed distally from the light source 32 via the optical fiber 28 toward the deployed stent 40. As previously described, the light is diffused adjacent the stent 40 to provide light exposure to the pre-polymer. Light is administered until the pre-polymer has nearly or fully polymerized or cured. The intensity, wavelength, and duration of light exposure can depend on factors such as, for example, the size of the elongate member 42 and amount of pre-polymer. Those skilled in the art will recognize that the strategy for diffusing and delivering the light energy to the pre-polymer may vary from the description and illustrations provided herein. For example, different pre-polymers and stent geometries may require alternative light sources from those described herein.

At step 450, the deployed stent is supported in a radial direction. In one embodiment, the polymerized polymer 60 serves to set the stent 40 in the deployed position and provide radial support. In another or the same embodiment, the biodegradable reinforcing member 50 provides additional support to the deployed stent 40. Once the photopolymerization is completed, balloon 30 is deflated and removed along with the catheter 20 and guidewire 26 leaving the deployed stent 40 at the treatment site.

At step 460, at least one therapeutic agent is eluted from the stent. In one embodiment, a single therapeutic agent may be integrated in any portion of the stent 40, such as the elongate member 42, reinforcing member 50, and/or polymer 60. In another embodiment, as previously described, two or more therapeutic agents are released with a multiple elution profile. Method 400 ends at step 470.

While the embodiments of the invention disclosed herein are presently considered to be preferred, various changes and modifications can be made without departing from the spirit and scope of the invention. For example, the elongate member, reinforcing member, and polymer are not limited to the illustrated and described embodiments. In addition, the method disclosed for treating a vascular condition may vary.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A biodegradable stent comprising:
a biodegradable flexible elongate member including an elongate member wall surrounding a cavity;
a biodegradable reinforcing member positioned within or adjacent the elongate member wall to support the biodegradable elongate member; and
a biodegradable photo-curable polymer positioned within the cavity of the elongate member.

2. The stent of claim 1 wherein at least one end portion of the elongate member is sealed.

3. The stent of claim 1 wherein the biodegradable elongate member comprises a coiled configuration.

4. The stent of claim 1 wherein the biodegradable photo-curable polymer provides support to the stent in a radial direction when in a deployed configuration.

5. The stent of claim 1 wherein the at least one therapeutic agent is eluted with a predetermined profile.

6. The stent of claim 1 wherein the reinforcing member comprises a magnesium wire.

7. The stent of claim 1 further comprising a photoinitiator positioned within the cavity.

8. An intraluminal stent delivery system comprising:
a catheter;
an inflatable member operably attached to the catheter;
a biodegradable stent disposed on the inflatable member, the stent comprising a biodegradable flexible elongate member including an elongate member wall surrounding a cavity, a biodegradable reinforcing member positioned within or adjacent the elongate member wall to support the biodegradable elongate member, and a biodegradable photo-curable polymer positioned within the cavity.

9. The system of claim 8 wherein at least one end portion of the elongate member is sealed.

10. The system of claim 8 wherein the reinforcing member comprises a coiled member.

11. The system of claim 8 wherein the biodegradable photo-curable polymer provides axial support to the stent.

12. The system of claim 8 wherein the at least one therapeutic agent is eluted with a predetermined profile.

13. The system of claim 8 further comprising at least one fiber optic member positioned adjacent the elongate member, wherein the fiber optic member is operably attached to a light source for polymerizing the photo-curable polymer.

14. The system of claim 13 wherein the at least one fiber optic member comprises a light diffusing member for radially diffusing light.

15. The system of claim 8 further comprising a photoinitiator positioned within the elongate member.

16. A method of treating a vascular condition, the method comprising:
delivering a biodegradable stent having a cavity filled with a pre-polymer to a treatment site;
expanding a balloon to position the stent at the treatment site;
photopolymerizing the pre-polymer positioned within the stent cavity; and
supporting the deployed stent in a radial direction with a biodegradable reinforcing member.

17. The method of claim 16 further comprising eluting at least one therapeutic agent from at least a portion of the stent.

18. The method of claim 17 wherein the at least one therapeutic agent is released with a predetermined profile.
